(19) United States
(12) Patent Application Publication
(10) Pub. No.: US 2005/0203356 A1
(43) Pub. Date: Sep. 15, 2005

(54) Joint-diagnostic in vivo & in vitro apparatus

(75) Inventor: James Samsoondar, Cambridge (CA)

Correspondence Address: BERESKIN AND PARR, 40 KING STREET WEST, BOX 401, TORONTO, ON, M5H 3Y2, CA

(73) Assignee: Chromedx Inc.

(21) Appl. No.: 11/071,247

(22) Filed: Mar. 4, 2005

(30) Foreign Application Priority Data
Mar. 9, 2004 (CA) 2,460,898

(51) Int. Cl. A61B 5/00; B65D 81/00

(52) U.S. Cl. 600/322; 600/573; 600/584; 128/903

(57) ABSTRACT

In vivo testing for analytes in a life-form is an attractive concept because a biological sample does not have to be removed from the life-form. However, in vivo testing alone is unable to provide information that is accurate, complete and/or reliable enough to safely replace in vitro testing. In contrast to performing either in vivo or in vitro testing independently and alone, some embodiments of the present invention provide a joint-diagnostic apparatus for combined in vivo and in vitro testing. In some specific embodiments results from an in vitro measurement module are used in combination with subsequent in vivo measurements/observations obtained at a later time, and/or vice versa. Accordingly, in some embodiments in vitro measurements are used to compliment and/or partially compensate for some of the limitations of in vivo testing, and at the same time enabling some of the benefits of in vivo testing by reducing the number of biological samples taken.

### PRIORITY CLAIM

This application claims the benefit of Canadian Patent Application No. 2,460,898 under the Paris Convention. The Canadian Patent Application No. 2,460,898 was filed on Mar. 9, 2004, and is hereby incorporated by reference.

### FIELD OF THE INVENTION

The invention relates to diagnostic testing for analytes in a life-form, and, in particular to apparatus for cooperative in vivo and in vitro testing.

### BACKGROUND OF THE INVENTION

In vitro testing for a particular analyte in a life-form (e.g. humans, wild and domestic animals, etc.) requires a biological sample to be taken from the life-form. That is, in vitro testing is an invasive procedure that involves removing something (e.g. blood, skin, organ tissue, etc.) from the life-form, thereby damaging the life-form. In addition to damaging the life-form, in vitro testing techniques have a number of inherent risks that cannot easily be avoided. For example, such risks include sample mix-up, infection of the life-form and those handling the biological samples, and loss of critical fluids and/or tissue. Pain is also sometimes an issue. Despite the risks, in vitro testing is often the only suitable way to accurately obtain diagnostic information about an analyte in a life-form.

By contrast, in vivo testing techniques are considered non-invasive because biological samples are not required. Generally, in vivo testing involves measuring things outside of a life-form without breaking the epidermis of the life-form and/or handling biological samples. A drawback to almost all of the known in vivo testing methods is that in vivo testing methods do not provide information that is either accurate or complete enough to enable reliable interpretation of the information actually gathered. Consequently, in vitro testing is often reverted to as a failsafe diagnostic tool, which negates the benefits provided by performing in vivo testing.

An example of a known in vivo testing method is disclosed in U.S. Pat. No. 6,654,622. Specifically, U.S. Pat. No. 6,654,622 discloses the measurement of carbon dioxide that has diffused through skin, and can be sensed by electrochemical detectors, in order to provide an assessment of the arterial oxygen saturation and the transcutaneous carbon dioxide partial pressure on an ear lobe. The system disclosed is incapable of determining blood pH, which is required to accurately interpret in vivo data when considering metabolic acidosis with respiratory compensation. As a result, blood must be drawn, which negates any benefit provided by the in vivo test, since all of the necessary information can be obtained from the blood sample.

Another example of an in vivo testing method is Near Infrared (NIR) spectroscopic analysis. The compartmentalization of body fluids presents currently unresolved complications for measuring a particular concentration of analytes in a life-form using NIR spectroscopic analysis. In particular, a NIR system cannot discriminate between analyte concentrations in different types of tissue/fluid, which means that a NIR system can only determine a tissue analyte concentration and not a blood (or another specific fluid) analyte concentration. The correlation between a particular blood (or another specific fluid) analyte concentration and a corresponding tissue analyte concentration is typically unreliable and has little predictive value. The errors produced from such data are referred to as “random inaccuracies”. Repetitive testing will not significantly reduce the magnitude of total error caused by random inaccuracies.

One specific example of an applied spectroscopic method is known as Pulse Oximetry. Pulse Oximetry can be used to monitor hemoglobin oxygen saturation or blood oxygen saturation (SaO₂) in a patient by measuring the light attenuated by a body part at two wavelengths—one wavelength selected from the visible spectrum and a second wavelength selected from the NIR spectrum. Successful Pulse Oximetry relies on the assumption that the Total-Hemoglobin (Tot-Hb) in a human is primarily composed of Oxy-hemoglobin (Oxy-Hb) and Deoxy-hemoglobin (Deoxy-Hb). However, this is not always a safe assumption, as there may be elevated levels of dyshemoglobins—such as, for example, Methemoglobin (Met-Hb), Carboxy-Hemoglobin (Carboxy-Hb) and Sulf-Hemoglobin (Su-Hb)—that are related to the condition of a patient. Increasing concentrations of dyshemoglobins cause Pulse Oximetry measurement errors to increase dramatically, leading to highly inaccurate measurements.

Another specific example of an applied spectroscopic method is described in U.S. Pat. No. 4,267,844. The U.S. Pat. No. 4,267,844 discloses a spectroscopic instrument for characterizing jaundice. Jaundice is caused by high levels of bilirubin and can lead to permanent brain damage in newborn babies (i.e. neonates). A reliable non-invasive method would be preferred to drawing blood from the neonates. However, spectroscopic measurement of bilirubin is complicated by the presence of bilirubin in more than one type of fluid/tissue and the unreliable correlation between bilirubin levels in blood (i.e., vascular tissue) and bilirubin levels in extra-vascular tissue.

### SUMMARY OF THE INVENTION

According to an aspect of an embodiment of the invention there is provided a joint-diagnostic apparatus including: an in vivo measurement module for analysis of a first analyte in a life-form; an in vitro measurement module for analysis of a second analyte in the life-form; and, a processor module having computer readable program code means embodied thereon for producing (i) a first parameter having a first value derived from the analysis of the first analyte, (ii) a second parameter having a second value derived from the analysis of the second analyte; and (iii) a combined result based on the first value and the second value.

In some embodiments the processor module comprises a Boolean operator for determining (i) if the first value meets an in vivo value threshold, (ii) the combined result to be the second value if the first value meets the in vivo value threshold, and (iii) the combined result to be a Boolean flag when the first value does not meet the in vivo value threshold.

In some embodiments, the processor module comprises a Boolean operator for determining (i) if the second value meets an in vitro value threshold, (ii) the combined result to be the first value if the second value meets the in vitro value threshold, and (iii) the combined result to be a Boolean flag when the second value does not meet the in vitro value threshold.

In some embodiments the first value is obtained by measuring a first measurable characteristic related to the first analyte, and the second value is obtained by measuring a second measurable characteristic related to the second analyte, and wherein the first observable characteristic differs from second observable characteristic.

In some embodiments the processor module comprises a computer readable program code means embodied therein for jointly analyzing the values of the first and second parameters, the computer readable program code means having computer readable instructions for determining a relationship between the first and second values.

In some embodiments the combined result includes a third parameter having a value related to the relationship between the first and second values. Alternatively, in other embodiments the first and second parameters do represent measurements of the same observable characteristic. Alternatively and/or additionally, a third value for the third parameter represents a measurement of the same observable characteristic as at least one of the first and second parameters. Alternatively and/or additionally, a third value for the third parameter represents a measurement of an observable characteristic different from both the first and second parameters.

In some embodiments the joint-diagnostic apparatus also includes: a remotely operable satellite device for collecting data; and a data-communication link for connecting the remotely operable satellite device to at least one of the in vivo measurement module, the in vitro measurement module, and the processor.

In some embodiments, the in vitro measurement module includes an electromagnetic radiation (EMR) source and detector for spectroscopic analysis. In some specific embodiments, the joint diagnostic apparatus also then includes a remotely operable satellite device defining a slot, and housing the EMR source and detector; and a data-communication link for connecting the remotely operable satellite device to at least one of the in vivo measurement module, the in vitro measurement module, and the processor.

According to some aspects of the invention, the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a measure of hemoglobin oxygen saturation (SaO₂) derived from the first parameter, and the computer readable program code means includes computer readable instructions for: determining respective relative amounts of different hemoglobin species present in the blood sample from the in vitro absorbance measurement of the blood sample; comparing each of the relative amounts of the different hemoglobin species present in the blood sample to a corresponding threshold value; and returning an indication about the measure of hemoglobin oxygen saturation (SaO₂) derived from the first parameter as a result of the comparison of each relative amount of hemoglobin species to its corresponding threshold value.

According to some other aspects of the invention the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a relative amount of Met-hemoglobin present in the life-form derived from the first parameter, and the computer readable program code means includes computer readable instructions for: determining a fourth parameter that is a relative amount of Met-hemoglobin present in the blood sample from the in vitro absorbance measurement of the blood sample; calculating a correction factor which is a ratio of the fourth parameter to the third parameter; and applying the correction factor to subsequent in vivo measurements of the third parameter.

According to yet other aspects of the invention the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a relative amount of Met-hemoglobin present in the life-form derived from the first parameter, and the computer readable program code means includes computer readable instructions for: determining a concentration of Methylene Blue in the blood sample from the in vitro absorbance measurement of the blood sample; comparing the concentration of Methylene Blue in the blood sample to a corresponding threshold value; and returning an indication about the concentration of Methylene Blue in the blood sample as a result of the comparison.

According to even other aspects of the invention the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a relative amount of hemoglobin-based blood substitute in Met-hemoglobin form present in the life-form derived from the first parameter, and the computer readable program code means includes computer readable instructions for: determining a fourth parameter that is a relative amount hemoglobin-based blood substitute in Met-hemoglobin form present in the blood sample from the in vitro absorbance measurement of the blood sample; calculating a correction factor which is a ratio of the fourth parameter to the third parameter; and applying the correction factor to subsequent in vivo measurements of the third parameter.

According to yet another aspect of the invention the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a relative amount of Carboxy-hemoglobin form present in the life-form derived from the first parameter, and the computer readable program code means includes computer readable instructions for: determining a fourth parameter that is a relative amount of Carboxy-hemoglobin present in the blood sample from the in vitro absorbance measurement of the blood sample; calculating a transforming factor which is a ratio of the fourth parameter to the third parameter; and applying the correction factor to subsequent in vivo measurements of the third parameter.

According to even yet another aspect of the invention the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a ratio of biliverdin and bilirubin present in the life-form derived from the first parameter, and the computer readable program code means includes computer readable instructions for: determining a fourth parameter that is a ratio of biliverdin and bilirubin present in the blood sample from the in vitro absorbance measurement of the blood sample; calculating a transforming factor which is a ratio of the fourth parameter to the third parameter; and applying the transforming factor to subsequent in vivo measurements of the third parameter.

According to another embodiment of the invention there is provided a joint-diagnostic in vivo and in vitro spectroscopic apparatus including: an in vivo spectroscopic measurement module for producing a first plurality of parameters, each having a value related to the spectroscopic analysis of a first plurality of analytes; an in vitro measurement module for producing a second plurality of parameters, each having a value related to the analysis of a second plurality of analytes; and a computer usable medium having computer readable program code means embodied therein for jointly analyzing some of each of the first and second pluralities of parameters thereby producing a third plurality of parameters from values of some of the first and second parameters, the third plurality of parameters being indicative of a clinical-relationship between some of the first and second pluralities of parameters.

Other aspects and features of the present invention will become apparent, to those ordinarily skilled in the art, upon review of the following description of the specific embodiments of the invention.

### BRIEF DESCRIPTION OF THE DRAWINGS

For a better understanding of the present invention, and to show more clearly how it may be carried into effect, reference will now be made, by way of example, to the accompanying drawings, which illustrate aspects of embodiments of the present invention and in which:

FIG. 1 is a schematic drawing of a first joint-diagnostic apparatus according to an embodiment of the invention;

FIG. 2 is a schematic drawing of a second joint-diagnostic apparatus according to an embodiment of the invention;

FIG. 3 is a schematic drawing of a third joint-diagnostic apparatus according to an embodiment of the invention;

FIG. 4 is a schematic drawing of a fourth joint-diagnostic apparatus according to an embodiment of the invention;

FIG. 5 is a schematic drawing of a fifth joint-diagnostic apparatus according to an embodiment of the invention;

FIG. 6 is a schematic drawing of a specific example of a remotely operable satellite device for use with a joint-diagnostic apparatus according to an embodiment of the invention;

FIG. 7 is a perspective view of an in vitro sample receptor suitable for use with a joint-diagnostic apparatus according to an embodiment of the invention;

FIG. 8 is an enlarged perspective view of a biological sample vessel shown in FIG. 7;

FIG. 9 is a flow chart illustrating a method of operating a joint-diagnostic apparatus, according to an aspect of the invention, to monitor hemoglobin oxygen saturation (SaO₂);

FIG. 10 is a flow chart illustrating a method of operating a joint-diagnostic apparatus, according to an aspect of the invention, to monitor oxidation of hemoglobin;

FIG. 11 is a flow chart illustrating a method of operating a joint-diagnostic apparatus, according to an aspect of the invention, to monitor methylene blue treatment of methemoglobinemia;

FIG. 12 is a flow chart illustrating a method of operating a joint-diagnostic apparatus, according to an aspect of the invention, to monitor oxidation of hemoglobin-based blood substitutes;

FIG. 13 is a flow chart illustrating a method of operating a joint-diagnostic apparatus, according to an aspect of the invention, to monitor fluctuating levels of Carboxy-hemoglobin;

FIG. 14 is a flow chart illustrating a method of operating a joint-diagnostic apparatus, according to an aspect of the invention, to monitor a ratio of biliverdin to bilirubin; and

FIG. 15 is a flow chart illustrating a method of operating a joint-diagnostic apparatus, according to an aspect of the invention, to monitor metabolic acidosis with respiratory compensation.

### DETAILED DESCRIPTION OF THE INVENTION

In vivo testing for analytes in a life-form is an attractive concept because a biological sample does not have to be removed from the life-form. However, in vivo testing alone is unable to provide information that is accurate, complete and/or reliable enough to safely replace in vitro testing in all circumstances. In contrast to performing either in vivo or in vitro testing independently and alone, some embodiments of the present invention provide a joint-diagnostic apparatus for cooperative in vivo and in vitro testing. In some specific embodiments results from an in vitro measurement module are used in combination with subsequent in vivo measurements/observations obtained at a later time to provide a combined result, and/or vice versa. Accordingly, in some embodiments in vitro measurements are used to compliment and/or partially compensate for some of the limitations of in vivo testing, and at the same time provide some of the benefits of in vivo testing by reducing the number of biological samples taken.

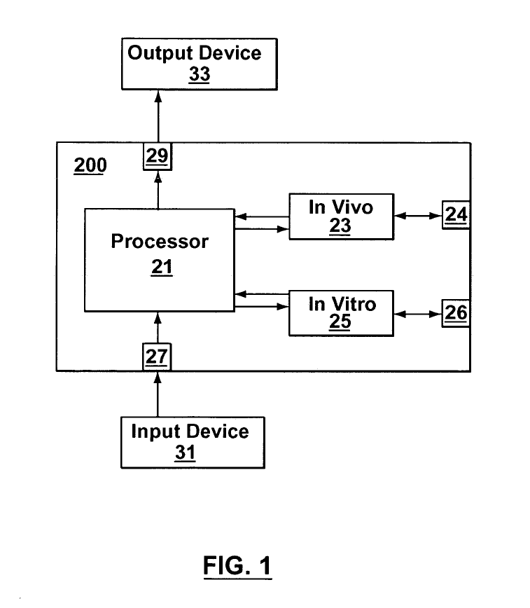

Referring to FIG. 1, shown is a schematic drawing of a joint-diagnostic apparatus 200 according to an embodiment of the invention. Those skilled in the art will appreciate that the joint-diagnostic apparatus 200 includes a suitable combination of structural elements, mechanical systems, hardware, firmware and software arranged to support the function and operation of the joint-diagnostic apparatus 200, and, for the sake of simplicity, portions of the joint-diagnostic apparatus 200 have been divided into functional units in order to conveniently describe aspects of this specific embodiment. To that end, the joint-diagnostic apparatus 200 includes a processor (i.e. processor module) 21, an in vivo measurement module 23 and an in vitro measurement module 25. The processor 21 is coupled to each of the in vivo and in vitro measurement modules 23, 25. Those skilled in the art will also appreciate that these functional blocks are not necessarily discrete elements in all embodiments of the invention.

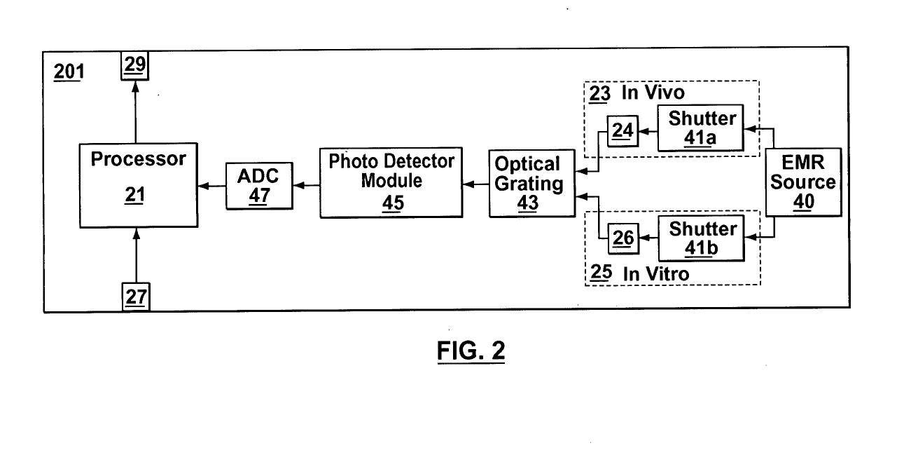

The joint diagnostic apparatus 200 also includes a number of data ports and receptors. The data ports provide data pathways to and from the processor 21, whereas the receptors provide measurement interfaces (i.e. sensing sites) for the in vivo and in vitro measurement modules 23, 25. Specifically, an input port 27 and an output port 29 are each coupled to the processor 21 to provide respective data paths to and from the processor 21. Respective in vivo and in vitro receptors 24 and 26 are provided as measurement interfaces for the corresponding in vivo and in vitro measurement modules 23 and 25. Examples of specific arrangements for the in vivo and in vitro receptors 24 and 26 are described in more detail further below with reference to FIGS. 2 and 5-8.

In some embodiments the input port 27 is coupled to receive instructions, input by a user, from an external input device 31. The external input device 31 may include, without limitation, a keyboard, a touch-pad/screen, a mouse, a digital-pen and a voice-command sensing system. Similarly, in some embodiments the output port 29 is coupled to deliver results, status-flags, printouts, warnings and the like to an external output device 33. The external output device 33 may include, without limitation, a display module, a printer, a hard-drive, another software program and an audible-signal generator. Additionally and/or alternatively, the input and output devices 31, 33 may be integrated into the joint-diagnostic apparatus 200.

In some embodiments the in vivo measurement module 23 includes, without limitation, electrochemical sensors, biosensors, spectroscopic devices and other types of transcutaneous sensors for non-invasively analyzing an analyte in a life form.

In some embodiments the in vitro measurement module 25 includes, without limitation, electrochemical sensors, biosensors, spectroscopic devices, chemical reagent delivery devices and other known devices used in in vitro testing to analyze an analyte in a biological sample taken from a life-form.

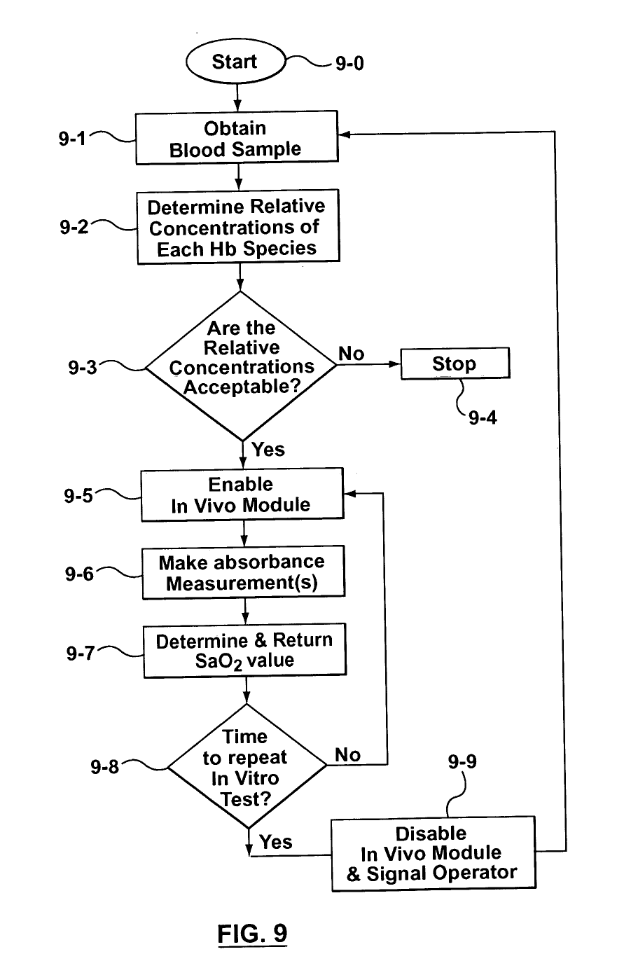

In operation the in vivo and in vitro measurement modules 23 and 25 are employed to analyze respective first and second analytes. The processor 21 has two alternative modes of operation. In both modes of operation the processor 21 generates a combined result from the analysis data/observations provided by the in vivo measurement module 23 and the in vitro measurement module 25. Accordingly, in some embodiments the processor 21 includes computer readable program code means embodied thereon for producing (i) a first parameter having a first value derived from the analysis of the first analyte, (ii) a second parameter having a second value derived from the analysis of the second analyte; and (iii) a combined result based on the first value and the second value. In the first mode of operation the combined result may serve as a Boolean flag, an agreement, a confirmation, a correction-factor, a transforming-factor and/or an integrity-assessment for the first value of the first parameter in light of the second value of the second parameter. In the second mode of operation the combined result may serve as a Boolean flag, an agreement, a confirmation, a correction-factor, a transforming-factor and/or an integrity-assessment for the second value of the second parameter in light of the first value of the first parameter. Specific examples of the two modes of operation, the parameters and analytes considered are described in detail below with reference to the flow-charts shown in FIGS. 9-15.

Referring now to FIG. 2, shown is a schematic drawing of a joint-diagnostic apparatus 201 arranged to perform spectroscopic analysis on a portion of a life-form and on a biological sample taken from the same life-form. Again, for the sake of simplicity, portions of the joint-diagnostic apparatus 201, shown in FIG. 2, have been divided into functional units in order to conveniently describe aspects of this specific embodiment of the invention. However, those skilled in the art will appreciate that the joint-diagnostic apparatus 201 also includes a suitable combination of associated structural elements, electrical systems, mechanical systems, hardware, firmware and software arranged to support the function and operation of the joint-diagnostic apparatus 201.

The joint-diagnostic apparatus 201 illustrated in FIG. 2 is similar to the joint-diagnostic apparatus 200 illustrated in FIG. 1, and accordingly, elements common to each are designated using the same reference numerals. For brevity, the description of FIG. 1 will not be repeated with respect to FIG. 2. Moreover, in addition to the features described with reference to FIG. 1, the joint-diagnostic apparatus 201 also includes an electromagnetic radiation (EMR) source 40, an optional optical grating 43, a photo-detector module 45 and an analog-to-digital converter (ADC) 47, that are provided so as to be shared by the in vivo and in vitro measurement modules 23, 25.

The optical grating 43, the photo-detector module 45 and the analog-to-digital converter (ADC) 47 are connected in series between the processor 21 and the in vivo and in vitro measurement modules 23, 25. In some embodiments the optical grating 43 includes a transmission grating and in other embodiments the optical grating 43 includes a reflection grating. In some embodiments, the photo-detector module 45 includes one or more photo-diodes arranged optionally into an array. However, other detectors, such as for example, Charged Coupled Detectors (CCD), may also be used.

The EMR source 40 is coupled to provide EMR to the in vivo and in vitro measurement modules 23, 25. In some embodiments the EMR source 40 is a tungsten lamp, a laser, a Light Emitting Diode (LED) or any combination thereof producing EMR at various desired wavelengths. The amount of EMR directed to each of the in vivo and in vitro measurement modules 23 and 25 can be fixed or variable as desired. In some embodiments the EMR 40 source is operable to produce EMR at at least one wavelength in the approximate range of 300 nm to 2500 nm.

Within the in vivo measurement module 23, a first shutter 41a is provided to control the flow of EMR into the in vivo receptor 24. Similarly, within the in vitro measurement module 25, a second shutter 41b is provided to control the flow of EMR into the in vitro receptor 26. The first and second shutters 41a and 41b are preferably independently controlled between open and closed positions, thereby adjusting the amount of EMR reaching the corresponding in vivo and in vitro receptors 24 and 26, respectively. In one specific mode of operation, the first shutter 41a is open and the second shutter 41b is fully closed, in order to perform in vivo analysis. In another specific mode of operation, the first shutter 41a is fully closed and the second shutter 41b is open, in order to perform in vitro analysis. As such, the control of the shutters 41a,b provides a multiplexing and/or toggle-control for the operation of the in vivo and in vitro measurement modules 23, 25.

Also in operation, the optical grating 43 receives a respective EMR output from one of the in vivo and in vitro measurement modules 23, 25 and the ADC 47 delivers electronic data derived from the spectroscopic outputs to the processor 21. The electronic data derived by the ADC 47 from the raw electrical output of the photo-detector module 45 is proportional to the time that the photo-detector module 45 integrates the spectroscopic signal received from the grating 43. Optionally, the electrical signal produced by the photo-detector module 45 may be amplified, attenuated, level shifted, and/or hard-limited. The electronic data derived by the ADC 47 is then processed by the processor 21 to produce absorbance data.

Provided for illustrative purposes only, equation (1), given below, has been derived as a very specific example of a quantitative approach to determining a numerical value for absorbance when conducting spectroscopic analysis.
Aᵢ=log{(RLᵢ−RDᵢ)/(SLᵢ−SDᵢ)}+log(ITS/ITR) (1)

Specifically, with respect to equation (1) the term Aᵢ is a measure of the absorbance by a pixel i, the terms RLᵢ and RDᵢ are the respective light and dark reference pixel readings for pixel i, the terms SLᵢ and SDᵢ are the respective light and dark sample pixel readings for pixel i, the term ITS is the integration time for the sample measurement, and the term ITR is the integration time for the reference measurement. A light sample reading refers to a pixel reading after exposure of the pixel to the EMR for a specific integration time. A dark reading refers to a pixel reading over the same integration time, with the pixel shielded from the EMR. It should be understood that the SLᵢ reading could be the EMR transmitted through the sample, the EMR reflected from the sample, or both. In some embodiments a form of absorbance pre-processing, for example, without limitation, taking the first derivative of absorbance, could be used in a calibration algorithm.

In alternative embodiments, the in vivo and in vitro measurement modules 23, 25 may be arranged to independently provide absorbance data that the processor 21 can use to produce a combined result. In such embodiments, each measurement module 23, 25 includes an optical grating, photo-detector, ADC and sub-processor.

Also, although the joint-diagnostic apparatus 201 illustrated in FIG. 2 appears to be a dual beam system, it is not because EMR in each module 23, 25 is ultimately directed through one of the respective receptors 24 and 26. That is, at any particular time the EMR from the EMR source 40 is directed only through in vivo or in vitro measurement receptors and not through a reference path that is independent of the state of each measurement receptor. Additionally and/or alternatively, one or both of the measurement modules 23, 25 may also include an EMR reference path (not shown), in which EMR does not travel through a receptor, but instead is used as a reference measurement, which provides compensation for fluctuations in the EMR, as shown in equation (1).

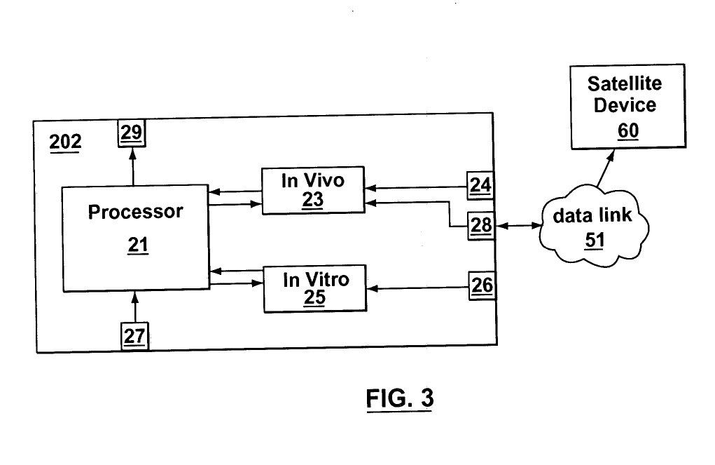

Referring now to FIG. 3, shown is a schematic drawing of another joint-diagnostic apparatus 202. Again, for the sake of simplicity, portions of the joint-diagnostic apparatus 202, shown in FIG. 3, have been divided into functional units in order to conveniently describe aspects of this specific embodiment of the invention. However, those skilled in the art will appreciate that the joint-diagnostic apparatus 202 also includes a suitable combination of associated structural elements, electrical systems, mechanical systems, hardware, firmware and software arranged to support the function and operation of the joint-diagnostic apparatus 202.

The joint-diagnostic apparatus 202 illustrated in FIG. 3 is similar to the joint-diagnostic apparatus 200 illustrated in FIG. 1, and accordingly, elements common to both share common reference numerals. For brevity, the description of FIG. 1 will not be repeated with respect to FIG. 3. In addition to those features described with reference to FIG. 1, the joint-diagnostic apparatus 202 includes a remotely operable satellite device 60 for collecting data relevant to the in vivo measurement module 23. In some embodiments, the satellite device 60 includes, without limitation, electrochemical sensors, biosensors and spectroscopic devices. Those skilled in the art will also appreciate that the satellite device 60 also includes a suitable combination of associated structural elements, electrical systems, mechanical systems, hardware, firmware and software arranged to support the function and operation of the satellite device 60.

The joint-diagnostic apparatus 202 also includes a communication port 28 that is internally coupled to the in vivo measurement module 23. In some embodiments, the communication port 28 and the satellite device 60 include, without limitation, simple wire connections, respective optical fiber modems, wireless modems, Universal Serial Bus (USB) ports, Ethernet modems or the like, in order to establish a data link 51. Accordingly, in operation, the communication port 28 is employed to establish the data link 51 between the in vivo measurement module 23 and the remotely operable satellite device 60.

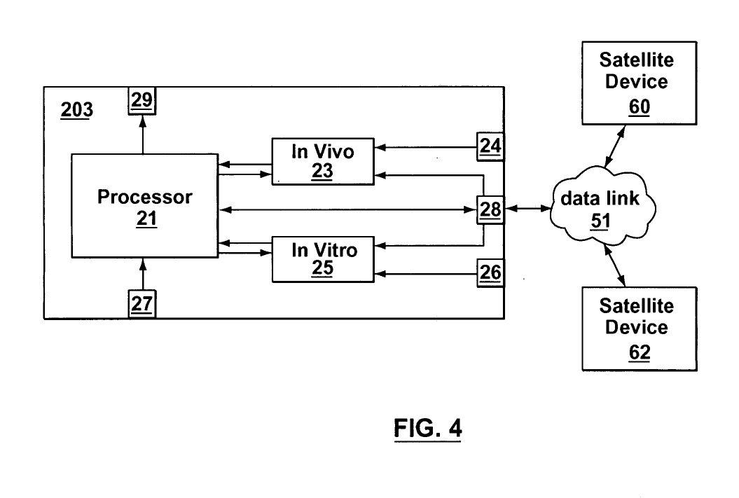

More generally, with continued reference to FIG. 3 and with now added reference to a joint-diagnostic apparatus 203 shown in FIG. 4, the communication port 28 may also be internally coupled to the in vitro measurement module 25 and the processor 21. Additionally and/or alternatively, the communication port 28 may be connected to any number of functional units within a joint-diagnostic apparatus according to an embodiment of the invention.

The joint-diagnostic apparatus 203, shown in FIG. 4, also includes a second remotely operable satellite device 62, which may or may not be similar to the satellite device 60. In some embodiments, the communication port 28 and the satellite devices 60 and 62 include, without limitation, simple wire connections, respective optical fiber modems, wireless modems, USB ports, Ethernet modems or the like, in order to establish the data link 51. In some embodiments the satellite devices 60, 62 include sensors (and optionally related elements) for collecting data relevant to the in vivo measurement module 23 and/or the in vitro measurement module 25. For example, the satellite device 62 can be used to remotely analyze a blood sample from within a quarantine testing-room, while the satellite device 60 is used to collect absorbance data for the in vivo measurement module 23. In some embodiments, the processor 21 and the in vivo and in vitro measurement modules 23, 25 share data collected from the satellite devices.

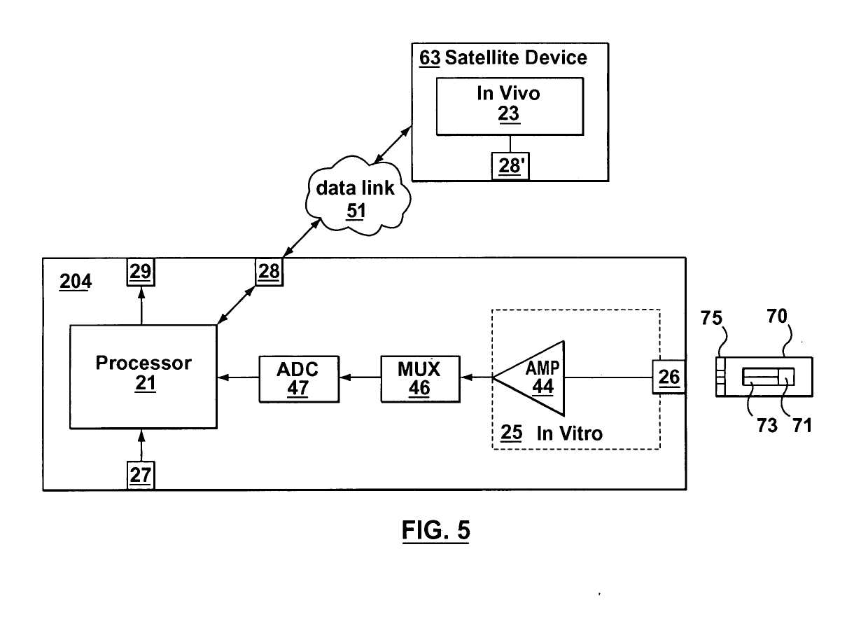

Referring to FIG. 5, shown is a schematic drawing of another joint-diagnostic apparatus 204 according to an embodiment of the invention. Again, for the sake of simplicity, portions of the joint-diagnostic apparatus 204, shown in FIG. 5, have been divided into functional units in order to conveniently describe aspects of this specific embodiment of the invention. However, those skilled in the art will appreciate that the joint-diagnostic apparatus 204 also includes a suitable combination of associated structural elements, electrical systems, mechanical systems, hardware, firmware and software arranged to support the function and operation of the joint-diagnostic apparatus 204.

The joint-diagnostic apparatus 204 illustrated in FIG. 5 is similar to the joint-diagnostic apparatus 200 illustrated in FIG. 1, and accordingly, elements common to both share common reference numerals. However, most of the in vivo measurement module 23 is now located in a remotely operable satellite device 63. Accordingly, in some embodiments, the satellite device 63 includes, without limitation, electrochemical sensors, biosensors and spectroscopic devices, in addition to a suitable combination of structural elements, electrical systems, mechanical systems, hardware, firmware and software arranged to support the function and operation of the satellite device 63.

The joint-diagnostic apparatus 204 includes a communication port 28 that is coupled to a multiplexer 46 (MUX 46, which is described in further detail below), and the satellite device 63 has a corresponding communication port 28′. In some embodiments, the communication ports 28, 28′ include, without limitation, simple wire connections, respective optical fiber modems, wireless modems, USB ports, Ethernet modems and the like, in order to establish the data link 51.

The in vitro measurement module 25 includes an amplifier 44 connected to the in vitro receptor 26 and a cartridge 70 that can be inserted into the in vitro receptor 26. The cartridge 70 includes a biological sample reservoir 71, one or more sensors 73 (two shown), and electrical contacts 75 (two shown). The sensors 73 may include, without limitation, electrochemical sensors, biosensors, chemically sensitive membranes, and the like, for analyzing a biological sample in the reservoir 71. The sensors 73 are coupled to electrical contacts 75 that are used to relay electrical signals from the sensors 73 to the in vitro receptor 26. In some embodiments, the in vitro receptor 26 includes at least one spectroscopic device.

Within the joint-diagnostic apparatus 204, the in vivo and in vitro measurement modules 23, 25 share common elements through the use of the multiplexer (MUX) 46. The communication port 28′, for the in vivo measurement module 23, and the amplifier 44, of the in vitro measurement module 25, are coupled to the multiplexer 46. The multiplexer 46 toggles access to the ADC 47, and ultimately the processor 21, between the in vivo and in vitro measurement modules 23, 25. In alternative embodiments, the in vivo and in vitro measurement modules 23, 25 may each include respective sub-processors and ADCs.

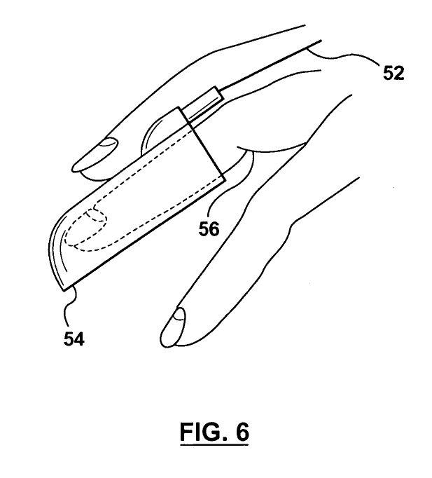

A schematic drawing of a finger receptor 54, according to an embodiment of the invention, is illustrated in FIG. 6. The finger receptor 54 is adapted to fit around a finger 56. The finger receptor 54 is a very specific example of a remotely operable satellite device that is suitable for use in various embodiments of the invention. The finger receptor 54 includes a data port 52 and at least one sensor (not shown). Optionally an EMR source, such as for example, one or more light emitting diodes (LEDs), may also be included in the finger receptor 54. The sensor may be, without limitation one of an electrochemical sensor, a biosensor, a spectroscopic sensor, a heat sensor, a pulse sensor, etc. More generally, a satellite device, such as the finger receptor 54, may include other elements included in one of or both of the in vivo and in vitro measurement modules 23, 25. In alternative embodiments, a satellite device, like the finger receptor 54, is adapted to fit around or be placed next to another body part.

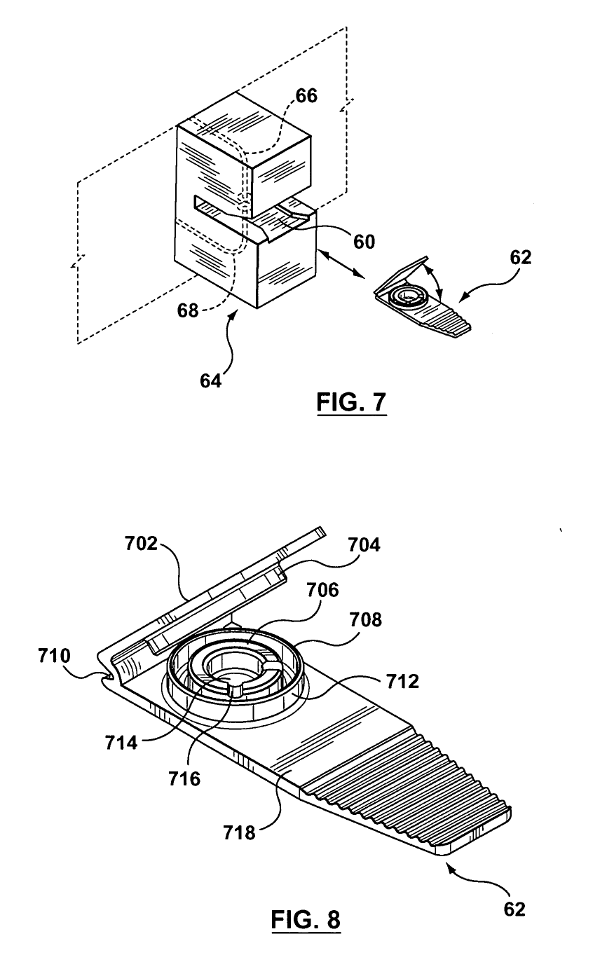

Referring now to FIGS. 7 and 8, shown are respective perspective views of an in vitro receptor 64 and a biological sample vessel 62 suitable for use with various embodiments of the invention. The in vitro receptor 64, shown in FIG. 7, is specifically provided to perform spectroscopic analysis on a biological sample contained in the biological sample vessel 62. The in vitro receptor 64 includes an input slot 60 into which the biological sample vessel 62 can be placed. Incident EMR is delivered into the slot 60 by an optical fiber 68. The EMR having passed through a biological sample is received by a receiving optical fiber 66. In alternative embodiments, an EMR source and detector can be arranged around the slot 60. In such embodiments, the optical fibers 66, 68 would not necessarily be required.

With specific reference to FIG. 8, the biological sample vessel 62 is a very specific example of a vessel that is suitable for use with various embodiments of the invention. The biological sample vessel 62 includes a base plate 718, a cover plate 702 and sample well 714 defined by closed wall 706. In some embodiments, a hinge 710 connects the cover plate 702 to the base plate 718 by a hinge 710. Alternatively, a non-hinged cover plate may also be used. In some such embodiments the cover plate 702 may be snapped on to the base plate 718. Overflow openings or grooves 716 in closed wall 706 allow excess sample to flow out of sample well 714 when cover plate 702 is closed over sample well 714 and base plate 718. A second containment wall 712 may be employed to retain the sample that overflows the sample well 714, into an overflow ring (circular groove between wall 706 and wall 712) to prevent leakage of fluid from the sample tab, while permitting a sample of sufficient volume to fill the well.

The biological sample vessel may be manufactured from a number of different materials, such as for example, but not limited to, a transparent, translucent or reflective material, such as glass, plastic or a combination thereof, or a reflective material. If the base plate 718 and cover plate 702 are transparent or translucent, then it is preferred that the base plate 718 and cover plate 702 is made from a transparent or translucent plastic, such as but not limited to polypropylene, polycarbonate, polyethylene, or polystyrene, however, a glass plate may also be used. If either of the base plate 718 or cover plate 702 is reflective, then a reflective material, for example but not limited to a ceramic coating, barium sulfate, Spectralon™, Spectraflect™, or Duraflect™ may be used for one of the base or cover plates 702, 718.

In alternative embodiments, items such as cuvettes, cartridges, test tubes, pipette tips, Petri dishes, beakers, flasks and the like could also be used to hold a biological sample.

Flow-charts are provided in FIGS. 9-15 that illustrate very specific examples of how a joint-diagnostic apparatus according to an embodiment of the invention can be applied to provide co-operative in vitro and in vivo testing using a single apparatus provided by an embodiment of the invention.

Specifically, referring to FIG. 9, shown is a flow chart depicting a method for monitoring hemoglobin (Hb) oxygen saturation (SaO₂) employing a joint-diagnostic apparatus according to an embodiment of the invention. At step 9-1, a blood sample is obtained from a patient. The blood sample is analyzed at step 9-2 in an in vitro measurement module to determine relative concentrations of different hemoglobin species present in the blood sample. In some embodiments, the in vitro analysis of the blood sample includes at least one of spectroscopic analysis, mechanical analysis, electrochemical analysis, biosensor analysis, chemical analysis with the aid of a reagent, and analysis without the aid of a reagent.

At step 9-3, at least one of the hemoglobin species is compared to a respective threshold value to determine whether or not valid and/or relatively accurate data can be obtained from subsequent in vivo measurements. If the at least one of the hemoglobin species violates a respective threshold (e.g. there is either too much or too little of the species present), then the joint-diagnostic apparatus disables an in vivo measurement module and signals the operator at step 9-4 (no path, step 9-3). On the other hand, if none of the thresholds is violated (yes path, step 9-3), the in vivo measurement module is enabled at step 9-5. Subsequently, at step 9-6 in vivo spectroscopic measurements are made, and a measurement of oxygen saturation is ultimately provided in step 9-7, using for example, without limitation, a pulse oximeter.

At step 9-8, it is determined whether or not an in vitro test should be repeated. Criteria for making such a determination include, without limitation, an amount of time elapsed since the last in vitro test, an upper limit for a number of in vivo tests that can be performed between in vitro testing, anomalous data provided by the in vivo testing, a lower threshold for hemoglobin oxygen saturation as determined by in vivo testing. If further in vitro testing is not required (no path, step 9-8), subsequent in vivo testing may be repeated starting from step 9-5. On the other hand, if further in vitro testing is deemed to be required (yes path, step 9-8), then the in vivo measurement module is disabled and the operator is signaled accordingly at step 9-9. As such, the method is repeated starting at step 9-1.

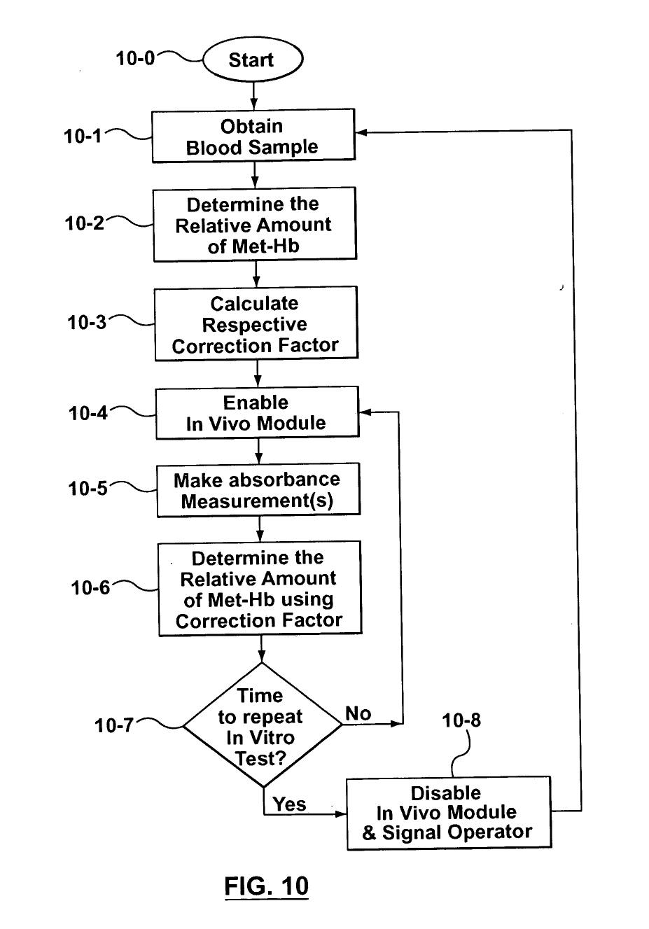

Referring now to FIG. 10, shown is a flow chart depicting a method for monitoring oxidation of hemoglobin (Hb) during treatment for methemoglobinemia, employing a joint-diagnostic apparatus according to an embodiment of the invention.

Oxidation of the iron in the heme moiety of Hb molecules is a normal process. Enzymes are continually at work reversing the oxidation process and thus preventing the accumulation of Met-Hb. Methemoglobinemia is a condition in people that lack enzymes required to reverse the oxidation process. In a specific example, the Met-Hb reductase system may be underdeveloped in infants, making methemoglobinemia more prevalent among infants. One specific reason for the higher incidence of methemoglobinemia among infants and neonates is an underdeveloped gastrointestinal system. In an underdeveloped gastrointestinal system, the bacteria level could rise due to a decreased secretion of gastric acid. Nitrates are usually converted into nitrites by bacteria of the gastrointestinal system, and the nitrites in turn react with the Hb to produce Met-Hb. Blood loss is critical in neonates, and even a heel-prick blood sample from a pre-matured neonate is considered critical blood loss. In these neonates, the decreased frequency of blood sampling due to the use of an in vivo apparatus that monitors % Met-Hb would be especially useful.

At step 10-1, a blood sample is obtained from a patient. The blood sample is analyzed at step 10-2 in an in vitro measurement module to determine a relative concentration of Met-Hemoglobin (Met-Hb) present in the blood sample. In some embodiments, the in vitro analysis of the blood sample includes at least one of spectroscopic analysis, mechanical analysis, electrochemical analysis, biosensor analysis, chemical analysis with the aid of a reagent, and analysis without the aid of a reagent.

At step 10-3, a correction factor, which can be used in combination with subsequent in vivo measurements, is calculated using the results of the in vitro analysis. Briefly, the correction factor is used to adjust subsequent in vivo measurements in view of the initial in vitro measurement. In some embodiments, the correction factor is calculated as a ratio of the relative amount of Met-Hb determined using in vitro analysis to a relative amount of Met-Hb determined using a preliminary in vivo spectroscopic measurement. The in vivo measurement module is then enabled at step 10-4, and further in vivo spectroscopic measurements are made at step 10-5. In alternative embodiments the relative amount of Met-Hb determined using in vitro analysis is employed in a regressive algorithm in which the correction factor is updated between each subsequent in vivo test. Subsequently, at step 10-6 the relative amount of Met-Hb can be derived using the correction factor previously determined.

At step 10-7, it is determined whether or not an in vitro test should be repeated. Criteria for making such a determination include, without limitation, an amount of time elapsed since the last in vitro test, an upper limit for a number of in vivo tests that can be performed between in vitro testing, anomalous data provided by the in vivo testing, and an upper or lower threshold for Met-Hb concentration as determined by in vivo testing. If further in vitro testing is not required (no path, step 10-7), subsequent in vivo testing may be repeated starting from step 10-4. On the other hand, if further in vitro testing is deemed to be required (yes path, step 10-7), then the in vivo measurement module is disabled and the operator is signaled accordingly at step 10-8. As such, the method is repeated starting at step 10-1.

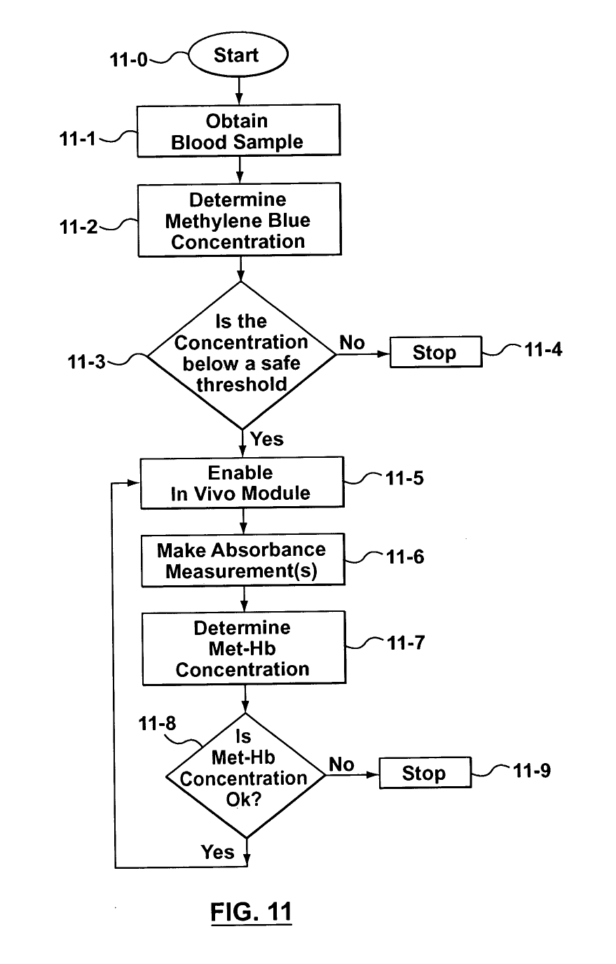

FIG. 11 is a flow chart illustrating a method for monitoring levels of methylene blue, in the treatment of methemoglobinemia, employing a joint-diagnostic apparatus according to an embodiment of the invention. Although methylene blue is used to treat methemoglobinemia, high doses have a paradox affect that in turn induces methemoglobinemia as opposed to reducing it. Therefore, it is important to maintain the blood concentration of methylene blue between an upper limit and a lower limit. The upper and lower limits together define a therapeutic window for the dosage of methylene blue. At step 11-1, a blood sample is obtained from a patient. The blood sample is analyzed at step 11-2 in an in vitro measurement module to determine a relative concentration of methylene blue. In some embodiments, the in vitro analysis of the blood sample includes at least one of spectroscopic analysis, mechanical analysis, electrochemical analysis, biosensor analysis, chemical analysis with the aid of a reagent, and analysis without the aid of a reagent.

At step 11-3, it is determined whether or not the amount of methylene blue violates a threshold (e.g. a therapeutic window). If the amount of methylene blue present in the blood sample violates the threshold, then the joint-diagnostic apparatus disables an in vivo measurement module and signals the operator at step 11-4 (no path, step 11-4). The signal to the operator may specifically indicate that the methylene blue level has reached a toxic level and/or that the in vitro measurement of Met-Hb level is abnormally high. On the other hand, if the threshold is not violated (yes path, step 11-3), the method proceeds to step 11-5, in which the in vivo measurement module is enabled. Subsequent in vivo absorbance measurements are made at step 11-6 from which a relative amount of Met-Hb can be derived at step 11-7.

At step 11-8, it is determined whether or not the Met-Hb level is within an acceptable range. If the Met-Hb level is not within an acceptable range (no path, step 11-8), in vivo testing is stopped and an operator is signaled accordingly at step 11-9. On the other hand, if the Met-Hb level is within an acceptable range further in vivo testing continues from step 11-5 (yes path, step 11-8).

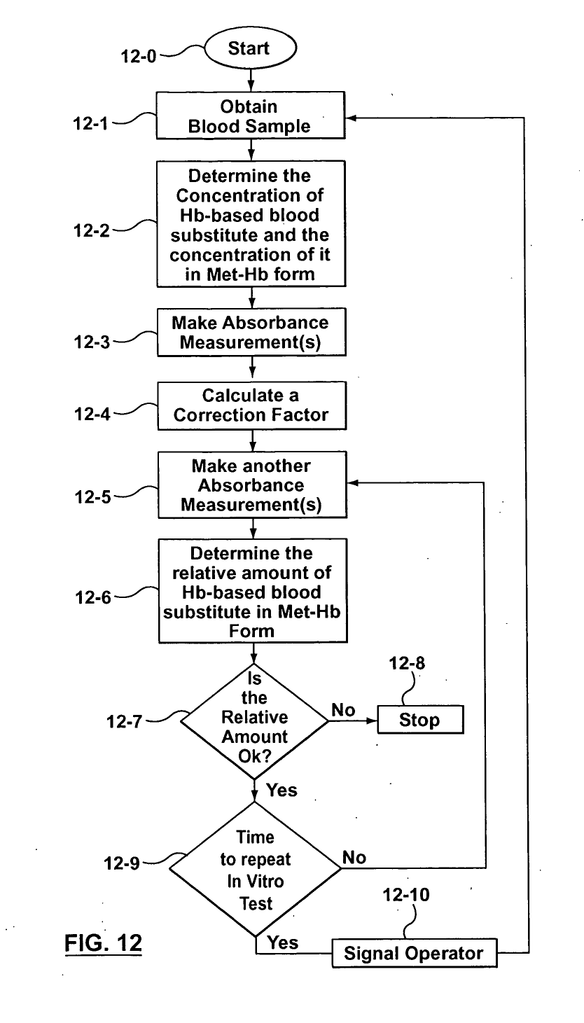

Referring to FIG. 12, shown is a flow chart depicting a method monitoring oxidation of hemoglobin-based blood substitutes. At step 12-1, a blood sample is obtained from a patient. The blood sample is analyzed at step 12-2 in an in vitro measurement module to determine concentrations of the Hb-based blood substitute and the amount of the Hb-based blood substitute in Met-Hb form. In some embodiments, the in vitro analysis of the blood sample includes at least one of spectroscopic analysis, mechanical analysis, electrochemical analysis, biosensor analysis, chemical analysis with the aid of a reagent, and analysis without the aid of a reagent.

A preliminary in vivo spectroscopic measurement is made at step 12-3. Then, at step 12-4, a correction factor, which can be used in combination with subsequent in vivo measurements, is calculated using the results of the in vitro analysis. In some embodiments, the correction factor is calculated as a ratio of the relative amount of Met-Hb determined using in vitro analysis to a relative amount of Met-Hb determined using the preliminary in vivo absorbance measurement. The in vivo measurement module is then enabled and further in vivo spectroscopic measurements are made at step 12-5. In alternative embodiments the relative amount of Met-Hb determined using in vitro analysis is employed in a regressive algorithm in which the correction factor is updated between each subsequent in vivo test. Subsequently, at step 12-6 the relative amount of Met-Hb can be derived using the correction factor previously determined.

At step 12-7, it is determined whether or not the amount of Met-Hb present violates a threshold (e.g. a safety threshold and/or upper or lower limit concentration). If the amount of Met-Hb violates the threshold, then the joint-diagnostic apparatus disables an in vivo measurement module and signals the operator at step 12-8 (no path, step 12-7). On the other hand, if the threshold is not violated (yes path, step 12-7), the method proceeds to step 12-9.

At step 12-9, it is determined whether or not an in vitro test should be repeated. Criteria for making such a determination include, without limitation, an amount of time elapsed since the last in vitro test, an upper limit for a number of in vivo tests that can be performed between in vitro testing, anomalous data provided by the in vivo testing, an upper or lower threshold for Met-Hb as determined by in vivo testing. The amount of Met-Hb may be quantified either as a concentration or as a fraction of total hemoglobin. If further in vitro testing is not required (no path, step 12-9), subsequent in vivo testing may be repeated starting from step 12-5. On the other hand, if further in vitro testing is deemed to be required (yes path, step 12-9), then the in vivo measurement module is disabled and the operator is signaled accordingly at step 12-10. As such, the method is repeated starting at step 12-1.

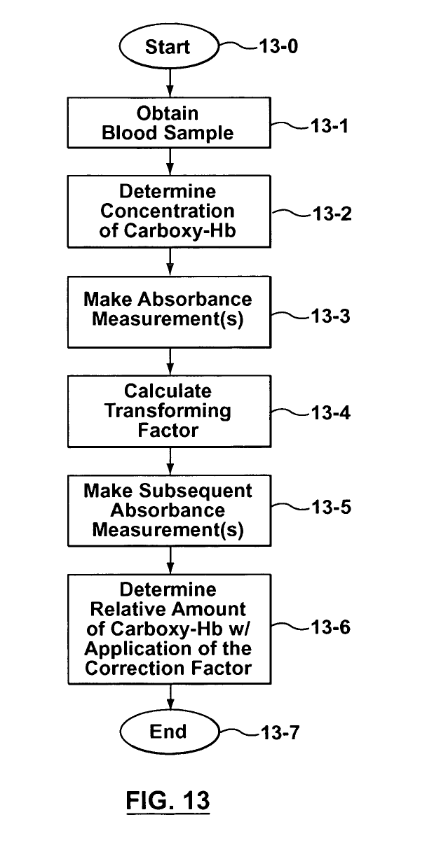

Yet another method of operating a joint-diagnostic apparatus is illustrated in FIG. 13. Specifically, FIG. 13 is a flow chart depicting a method of monitoring fluctuating levels of Carboxy-Hemoglobin (Carboxy-Hb).

A measurement of the ratio of Carboxy-Hb to Total-Hb could be used for monitoring the accumulation of Carboxy-Hb after exposure to carbon monoxide, and could also be used for monitoring the re-conversion of Carboxy-Hb into Oxy-Hb, after optional treatment with oxygen. Unlike Oxy-Hb, which exists in significantly different levels in arterial and venous blood, Carboxy-Hb exists in approximately the same levels in all blood compartments. Hence, the in vivo Carboxy-Hb measurement does not have to be synchronized with the pulse as in pulse oximetry. On the other hand, whereas a calibration algorithm for hemoglobin oxygen saturation can be developed empirically by administering oxygen to human subjects, it is unethical to do the same with carbon monoxide.

At step 13-1, a blood sample is obtained from a patient. The blood sample is analyzed at step 13-2 in an in vitro measurement module to determine a relative concentration of Carboxy-Hb in the blood sample. In some embodiments, the in vitro analysis of the blood sample includes at least one of spectroscopic analysis, mechanical analysis, electrochemical analysis, biosensor analysis, chemical analysis with the aid of a reagent, and analysis without the aid of a reagent. A preliminary in vivo spectroscopic measurement is made at step 13-3.

At step 13-4, a transforming factor, which can be used in combination with subsequent in vivo measurements, is calculated using the results of the in vitro analysis. In some embodiments, the transforming factor is determined from a ratio of the relative amount of Carboxy-Hb determined using in vitro analysis to a preliminary in vivo spectroscopic measurement. The transforming factor is used to convert the absorbance measurement into a measurement of Carboxy-HB. The in vivo measurement module is then enabled and further in vivo spectroscopic measurements are made at step 13-5. Subsequently, at step 13-6 the relative amount of Carboxy-Hb can be derived using a correction factor previously determined as described below for a alternative embodiment. Those skilled in the art will appreciate that steps 13-4 to 13-6 can be repeated a number of times between subsequent blood samples and in vitro testing as described above.

In an alternative embodiment a preliminary in vivo calibration algorithm for the relative amount of Carboxy-Hb is developed using spectroscopic data and corresponding blood Carboxy-Hb data, previously collected from other patients and/or the present patient being monitored. A correction factor is determined from the ratio of the Carboxy-Hb determined by the preliminary in vivo calibration algorithm and the Carboxy-Hb determined by the in vitro.

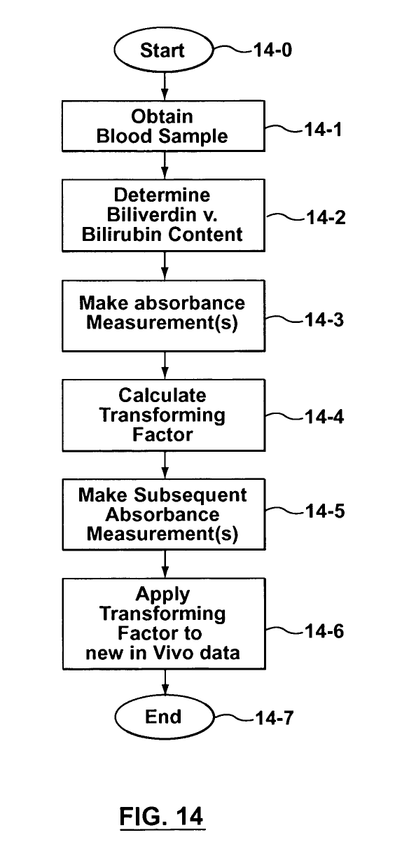

Referring to FIG. 14, shown is a flow chart depicting a method of monitoring relative amounts of biliverdin to bilirubin. At step 14-1, a blood sample is obtained from a patient. The blood sample is analyzed at step 14-2 in an in vitro measurement module to determine a ratio of biliverdin to bilirubin in the blood sample. In some embodiments, the in vitro analysis of the blood sample includes at least one of spectroscopic analysis, mechanical analysis, electrochemical analysis, biosensor analysis, chemical analysis with the aid of a reagent, and analysis without the aid of a reagent. A preliminary in vivo absorbance measurement is made at step 14-3. Then, at step 14-4, a transforming factor, which can be used in combination with subsequent in vivo measurements, is calculated using the results of the in vitro analysis. In some embodiments, the transforming factor is calculated as a ratio of the ratio of biliverdin to bilirubin determined using in vitro analysis to that same ratio determined using the preliminary in vivo absorbance measurement. The in vivo measurement module is then enabled and further in vivo EMR absorbance measurements are made at step 14-5.

In alternative embodiments the ratio of biliverdin to bilirubin determined using in vitro analysis is employed in a regressive algorithm in which a correction factor is determined and updated between each subsequent in vivo test. Subsequently, at step 14-6 the ratio of biliverdin to bilirubin can be derived using the correction factor previously determined. Those skilled in the art will appreciate that steps 14-4 to 14-6 can be repeated a number of times between subsequent blood samples and in vitro testing as described above.

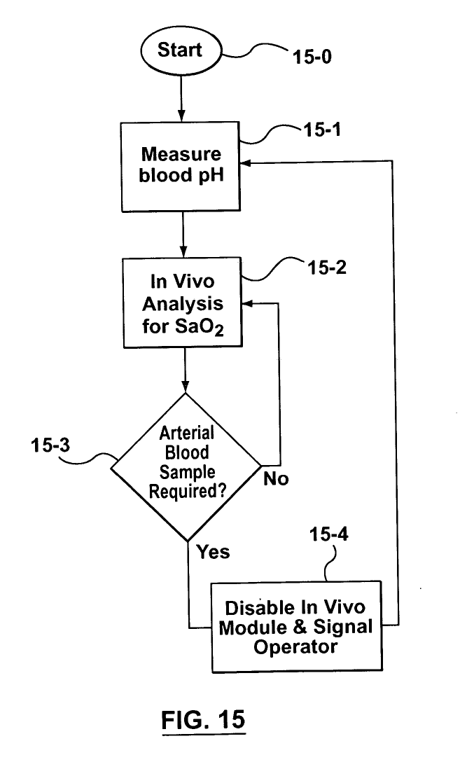

Lastly, referring to FIG. 15 shown is a flow chart illustrating a method for conducting diagnostic measurements related to blood gas measurement, which usually includes partial pressures of oxygen and carbon dioxide in blood, and blood pH. At step 15-1, an in vitro measurement module is employed to measure the blood pH in a pin-prick blood sample (capillary blood). Following the determination of blood pH, at step 15-2 an assessment of the arterial oxygen saturation and the transcutaneous carbon dioxide partial pressure is made. This may be accomplished in the same manner as described in U.S. Pat. No. 6,654,622. Transcutaneous carbon dioxide partial pressure measurement is considered non-invasive since the skin does not have to be broken in order to obtain a result.

At step 15-3, it is determined whether or not an arterial blood sample must be obtained for blood gas measurement. Criteria for making such a determination include, without limitation, discordance between the pH in the capillary blood sample and the transcutaneous carbon dioxide partial pressure. In one specific example, a discordance between blood pH and the transcutaneous carbon dioxide partial pressure can lead to a false-diagnosis of alkalosis, which occurs when the blood pH is too high. By contrast acidosis (respiratory or metabolic) is a condition manifested by low blood pH. High transcutaneous partial pressure of carbon dioxide is also indicative of acidosis, in particular, respiratory acidosis. However, when a person has metabolic acidosis, the body's mechanism to counteract the acidosis is respiratory compensation (i.e. hyperventilation). This condition is called metabolic acidosis with respiratory compensation, and in such a condition, the transcutaneous carbon dioxide partial pressure may be low and can be interpreted as alkalosis.

If an arterial blood sample is not required (no path, step 15-3), subsequent in vivo testing may be repeated starting from step 15-2. On the other hand, if an arterial blood sample is required (yes path, step 15-3), then the in vivo measurement module is disabled and the operator is signaled accordingly at step 15-4. As such, the method is repeated from step 15-1.

While the above description provides example embodiments, it will be appreciated that the present invention is susceptible to modification and change without departing from the fair meaning and scope of the accompanying claims. Accordingly, what has been described is merely illustrative of the application of aspects of embodiments of the invention. Numerous modifications and variations of the present invention are possible in light of the above teachings. It is therefore to be understood that within the scope of the appended claims, the invention may be practiced otherwise than as specifically described herein.

1. A joint-diagnostic apparatus comprising:
an in vivo measurement module for analysis of a first analyte in a life-form;
an in vitro measurement module for analysis of a second analyte in the life-form; and,
a processor module having computer readable program code means embodied thereon for producing (i) a first parameter having a first value derived from the analysis of the first analyte, (ii) a second parameter so having a second value derived from the analysis of the second analyte; and
(iii) a combined result based on the first value and the second value.

2. An apparatus according to claim 1 further comprising a display module for displaying the combined result.

3. An apparatus according to claim 1 wherein the processor module comprises a Boolean operator for determining (i) if the first value meets an in vivo value threshold, (ii) the combined result to be the second value if the first value meets the in vivo value threshold, and (iii) the combined result to be a Boolean flag when the first value does not meet the in vivo value threshold.

4. An apparatus according to claim 1 wherein the processor module comprises a Boolean operator for determining (i) if the second value meets an in vitro value threshold, (ii) the combined result to be the first value if the second value meets the in vitro value threshold, and (iii) the combined result to be a Boolean flag when the second value does not meet the in vitro value threshold.

5. An apparatus according to claim 1, wherein the first and second analytes are the same.

6. An apparatus according to claim 1, wherein the first value is obtained by measuring a first measurable characteristic related to the first analyte, and the second value is obtained by measuring a second measurable characteristic related to the second analyte, and wherein the first observable characteristic differs from second observable characteristic.

7. An apparatus according to claim 1, wherein the processor module comprises a computer readable program code means embodied therein for jointly analyzing the values of the first and second parameters, the computer readable program code means having computer readable instructions for determining a relationship between the first and second values.

8. An apparatus according to claim 7, wherein the combined result includes a third parameter having a value related to the relationship between the first and second values.

9. An apparatus according to claim 1, wherein the first and second parameters do represent measurements of the same observable characteristic.

10. An apparatus according to claim 8, wherein a third value for the third parameter represents a measurement of the same observable characteristic as at least one of the first and second parameters.

11. An apparatus according to claim 8, wherein a third value for the third parameter represents a measurement of an observable characteristic different from both the first and second parameters.

12. An apparatus according to claim 8, wherein the value of the third parameter is indicative of one of an agreement, a confirmation, a correction-factor and an integrity-assessment of the first value in view of the second value.

13. An apparatus according to claim 8, wherein the value of the third parameter is indicative of one of an agreement, a confirmation, a correction-factor and an integrity-assessment of the second value in view of the first value.

14. An apparatus according to claim 7 further comprising:
a remotely operable satellite device for collecting data; and
a data-communication link for connecting the remotely operable satellite device to at least one of the in vivo measurement module, the in vitro measurement module, and the processor.

15. An apparatus according to claim 14, wherein the data-communication link includes at least one of a wireless modem, a USB port, an Ethernet modem, a fiber-optic modem, a twisted-pair modem and a wire connection.

16. An apparatus according to claim 15, wherein the data-communication link is also operable to transmit data to the remotely operable satellite device.

17. An apparatus according to claim 14, wherein a portion of the in vivo spectroscopic measurement module is included within the remotely operable satellite device.

18. An apparatus according to claim 14, wherein a portion of the in vitro measurement module is included within the remotely operable satellite device.

19. An apparatus according to claim 14, wherein a portion of the processor is included within the remotely operable satellite device.

20. An apparatus according to claim 7, wherein the in vivo measurement module includes a receptor for accepting a portion of the life-form for spectroscopic analysis.

21. An apparatus according to claim 20, wherein the in vivo measurement module includes an electromagnetic radiation (EMR) source and detector arranged within the receptor.

22. An apparatus according to claim 21, wherein the detector is arranged to detect EMR that has travelled through the portion of the life-form accepted into the receptor.

23. An apparatus according to claim 21, wherein the detector is arranged to detect EMR that has reflected from the portion of the life-form accepted into the receptor.

24. An apparatus according to claim 21 further comprising:
a remotely operable satellite device housing the receptor, the EMR source and detector; and
a data-communication link for connecting the remotely operable satellite device to at least one of the in vivo measurement module, the in vitro measurement module, and the processor.

25. An apparatus according to claim 21, wherein the EMR source is operable to produce EMR at at least one wavelength in the approximate range of 300 nm to 2500 nm.

26. An apparatus according to claim 21, wherein the EMR source includes at least one of a tungsten lamp, a laser and a Light Emitting Diode (LED).

27. An apparatus according to claim 7, wherein the in vitro measurement module includes a receptor for accepting a biological sample from the life-form for analysis.

28. An apparatus according to claim 27, wherein the receptor is arranged to accept a vessel containing a biological sample.

29. An apparatus according to claim 28, wherein the vessel contains at least one reagent in combination with the biological sample.

30. An apparatus according to claim 29, wherein the biological sample includes at least one of whole blood, a portion of whole blood, interstitial fluid, serum, plasma, urine, cerebrospinal fluid, sputum, synovial fluid, lymphatic fluid and feces.

31. An apparatus according to claim 7, wherein the in vitro measurement module includes an electromagnetic radiation (EMR) source and detector for spectroscopic analysis.

32. An apparatus according to claim 31, wherein the detector is arranged to detect EMR that has travelled through the biological sample.

33. An apparatus according to claim 31, wherein the detector is arranged to detect EMR that has reflected from the biological sample.

34. An apparatus according to claim 31 further comprising:
a remotely operable satellite device defining a slot, and housing the EMR source and detector; and
a data-communication link for connecting the remotely operable satellite device to at least one of the in vivo measurement module, the in vitro measurement module, and the processor.

35. An apparatus according to claim 31, wherein the EMR source is operable to produce EMR at at least one wavelength in the approximate range of 300 nm to 2500 nm.

36. An apparatus according to claim 31, wherein the EMR source includes at least one of a tungsten lamp, a laser and a Light Emitting Diode (LED).

37. An apparatus according to claim 8, wherein the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a measure of hemoglobin oxygen saturation (SaO₂) derived from the first parameter, and the computer readable program code means includes computer readable instructions for:
determining respective relative amounts of different hemoglobin species present in the blood sample from the in vitro absorbance measurement of the blood sample;
comparing each of the relative amounts of the different hemoglobin species present in the blood sample to a corresponding threshold value; and
returning an indication about the measure of hemoglobin oxygen saturation (SaO₂) derived from the first parameter as a result of the comparison of each relative amount of hemoglobin species to its corresponding threshold value.

38. An apparatus according to claim 37, wherein the indication is at least one of an error message, a confirmation message, and a signal that disables the in vivo measurement module.

39. An apparatus according to claim 37, wherein the computer readable program code means also includes computer readable instructions for disabling the in vivo measurement module until an in vitro measurement is repeated.

40. An apparatus according to claim 39, wherein the instructions for disabling the in vivo measurement module, until an in vitro measurement is repeated, occurs after at least one of a predetermined number of in vivo measurements, a predetermined amount of time and if the measure of hemoglobin oxygen saturation (SaO₂) derived from the first parameter breaches a corresponding threshold value.

41. An apparatus according to claim 37, wherein the computer readable program code means also includes computer readable instructions for deriving the measure of hemoglobin oxygen saturation (SaO₂) from the first parameter according to a Pulse Oximetry technique that employs the use of at least two wavelengths of EMR.

42. An apparatus according to claim 8, wherein the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a relative amount of Met-hemoglobin present in the life-form derived from the first parameter, and the computer readable program code means includes computer readable instructions for:
determining a fourth parameter that is a relative amount of Met-hemoglobin present in the blood sample from the in vitro absorbance measurement of the blood sample;
calculating a correction factor which is a ratio of the fourth parameter to the third parameter; and
applying the correction factor to subsequent in vivo measurements of the third parameter.

43. An apparatus according to claim 42, wherein the relative amounts of Met-hemoglobin are calculated as a ratio of the amount of Met-hemoglobin to the total-hemoglobin.

44. An apparatus according to claim 42, wherein the computer readable program code means also includes computer readable instructions for disabling the in vivo measurement module until an in vitro measurement is repeated.

45. An apparatus according to claim 44, wherein the instructions for disabling the in vivo measurement module, until an in vitro measurement is repeated, occurs after at least one of a predetermined number of in vivo measurements, a predetermined amount of time and if the relative amount Met-hemoglobin derived from the first parameter breaches a corresponding threshold value in subsequent in vivo measurements.

46. An apparatus according to claim 42, wherein the computer readable program code means also includes computer readable instructions for deriving the relative amount of Met-hemoglobin from a ratio of absorbance measurements at two wavelengths.

47. An apparatus according to claim 8, wherein the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a relative amount of Met-hemoglobin present in the life-form derived from the first parameter, and the computer readable program code means includes computer readable instructions for:
determining a concentration of Methylene Blue in the blood sample from the in vitro absorbance measurement of the blood sample;
comparing the concentration of Methylene Blue in the blood sample to a corresponding threshold value; and
returning an indication about the concentration of Methylene Blue in the blood sample as a result of the comparison.

48. An apparatus according to claim 47, wherein the indication is at least one of an error message, a confirmation message, a toxicity measurement, the concentration of Methylene Blue, the time the in vitro measurement was made and a signal that disables the in vivo measurement module.

49. An apparatus according to claim 47, wherein the computer readable program code means also includes computer readable instructions for disabling the in vivo measurement module until an in vitro measurement is repeated.

50. An apparatus according to claim 49, wherein the instructions for disabling the in vivo measurement module, until an in vitro measurement is repeated, occurs after at least one of a predetermined number of in vivo measurements, a predetermined amount of time, and if the relative amount Met-hemoglobin derived from the first parameter breaches a corresponding threshold value in subsequent in vivo measurements.

51. An apparatus according to claim 47, wherein the computer readable program code means also includes computer readable instructions for deriving the relative amount of Met-hemoglobin from a ratio of absorbance measurements at two wavelengths.

52. An apparatus according to claim 8, wherein the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a relative amount of hemoglobin-based blood substitute in Met-hemoglobin form present in the life-form derived from the first parameter, and the computer readable program code means includes computer readable instructions for:
determining a fourth parameter that is a relative amount hemoglobin-based blood substitute in Met-hemoglobin form present in the blood sample from the in vitro absorbance measurement of the blood sample;
calculating a correction factor which is a ratio of the fourth parameter to the third parameter; and
applying the correction factor to subsequent in vivo measurements of the third parameter.

53. An apparatus according to claim 52, wherein the relative amounts of hemoglobin-based blood substitute in Met-hemoglobin form are calculated as a ratio of a concentration of hemoglobin-based blood substitute and a concentration of hemoglobin-based blood substitute in Met-hemoglobin form.

54. An apparatus according to claim 52, wherein the computer readable program code means also includes computer readable instructions for disabling the in vivo measurement module until an in vitro measurement is repeated.

55. An apparatus according to claim 54, wherein the instructions for disabling the in vivo measurement module, until an in vitro measurement is repeated, occurs after at least one of a predetermined number of in vivo measurements, a predetermined amount of time, and if the relative amount hemoglobin-based blood substitute in Met-hemoglobin form derived from the first parameter breaches a corresponding threshold value in subsequent in vivo measurements.

56. An apparatus according to claim 52, wherein the computer readable program code means also includes computer readable instructions for deriving the relative amount of hemoglobin-based blood substitute in Met-hemoglobin form from a ratio of absorbance measurements at two wavelengths.

57. An apparatus according to claim 8, wherein the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a relative amount of Carboxy-hemoglobin form present in the life-form derived from the first parameter, and the computer readable program code means includes computer readable instructions for:
determining a fourth parameter that is a relative amount of Carboxy-hemoglobin present in the blood sample from the in vitro absorbance measurement of the blood sample;
calculating a transforming factor which is a ratio of the fourth parameter to the third parameter; and
applying the correction factor to subsequent in vivo measurements of the third parameter.

58. An apparatus according to claim 57, wherein the relative amounts of Carboxy-hemoglobin are calculated as a ratio of a concentration of total-hemoglobin and a concentration of Carboxy-hemoglobin.

59. An apparatus according to claim 57, wherein the computer readable program code means also includes computer readable instructions for disabling the in vivo measurement module until an in vitro measurement is repeated.

60. An apparatus according to claim 59, wherein the instructions for disabling the in vivo measurement module, until an in vitro measurement is repeated, occurs after at least one of a predetermined number of in vivo measurements, a predetermined amount of time, and if the relative amount Carboxy-hemoglobin derived from the first parameter breaches a corresponding threshold value in subsequent in vivo measurements.

61. An apparatus according to claim 57, wherein the computer readable program code means also includes computer readable instructions for deriving the relative amount of Carboxy-hemoglobin from a ratio of absorbance measurements at two wavelengths.

62. An apparatus according to claim 8, wherein the first parameter is an in vivo absorbance measurement of a body part of the life-form taken at at least one wavelength of electromagnetic radiation (EMR), the second parameter is an in vitro absorbance measurement of a blood sample of the life form taken at at least one wavelength of EMR, the third parameter is a ratio of biliverdin and bilirubin present in the life-form derived from the first parameter, and the computer readable program code means includes computer readable instructions for:
determining a fourth parameter that is a ratio of biliverdin and bilirubin present in the blood sample from the in vitro absorbance measurement of the blood sample;
calculating a transforming factor which is a ratio of the fourth parameter to the third parameter; and
applying the transforming factor to subsequent in vivo measurements of the third parameter.

63. An apparatus according to claim 62, wherein the ratios of biliverdin and bilirubin are calculated from a concentration of biliverdin and a concentration of bilirubin.

64. An apparatus according to claim 62, wherein the computer readable program code means also includes computer readable instructions for disabling the in vivo measurement module until an in vitro measurement is repeated.

65. An apparatus according to claim 64, wherein the instructions for disabling the in vivo measurement module, until an in vitro measurement is repeated, occurs after at least one of a predetermined number of in vivo measurements, a predetermined amount of time, and if the third parameter breaches a corresponding threshold value in subsequent in vivo measurements.

66. An apparatus according to claim 62, wherein the computer readable program code means also includes computer readable instructions for deriving the ratio of biliverdin and bilirubin from a ratio of absorbance measurements at two wavelengths.

67. A joint-diagnostic in vivo and in vitro spectroscopic apparatus comprising:
an in vivo spectroscopic measurement module for producing a first plurality of parameters, each having a value related to the spectroscopic analysis of a first plurality of analytes;
an in vitro measurement module for producing a second plurality of parameters, each having a value related to the analysis of a second plurality of analytes; and,
a computer usable medium having computer readable program code means embodied therein for jointly analyzing some of each of the first and second pluralities of parameters thereby producing a third plurality of parameters from values of some of the first and second parameters, the third plurality of parameters being indicative of a clinical-relationship between some of the first and second pluralities of parameters.
